(19) United States
(12) Patent Application Publication
(10) Pub. No.: US 2023/0000659 A1
(43) Pub. Date: Jan. 5, 2023

(54) WRIST GUARD

(71) Applicant: Yoon Jin HWANG, Hanam-si (KR)

(72) Inventors: Yoon Jin HWANG, Hanam-si (KR); Young Gi KIM, Seoul (KR); Young Whan YU, Seoul (KR); Ji O LEE, Seoul (KR); Ji Won KANG, Seoul (KR)

(73) Assignee: Yoon Jin HWANG, Hanam-si (KR)

(21) Appl. No.: 17/856,396

(22) Filed: Jul. 1, 2022

(30) Foreign Application Priority Data
Jul. 1, 2021 (KR) 10-2021-0086582

(51) Int. Cl. A61F 5/058

(52) U.S. Cl. CPC A61F 5/05866

(57) ABSTRACT

A wrist guard includes: a compression part having elasticity and surrounding the periphery of the wrist; a band part disposed on the outer side of the compression part to surround and tighten the circumference of the compression part surrounding the wrist; and a reinforcing bar disposed between the band part and the compression part, located around the ligament covering the wrist tunnel, and extending toward the elbow.

### CROSS-REFERENCE TO RELATED APPLICATION

This application claims priority to and the benefit of Korean Patent Application No. 10-2021-0086582 filed on Jul. 1, 2021, which is hereby incorporated by reference in its entirety.

### BACKGROUND

The present invention relates to a wrist guard, and more particularly, to a wrist guard which intensively reinforces the wrist tunnel and the periphery of the ligament covering the wrist tunnel which causes pain when the wrist is bent, thereby enhancing a reinforcing force to the wrist of a user and preventing inconvenience when the user bends the wrist.

In general, the wrist performs an important role to move the hand or grip a heavy object with the hand in real life. When a person strains the wrist, it may cause the carpal tunnel syndrome.

The carpal tunnel syndrome is compression neuropathy, and is caused by median nerve compression or median nerve entrapment occurring in the wrist joint.

That is, the carpal tunnel syndrome is a nerve disease in which the carpal tunnel forming a small passage formed by the bone and the ligaments of the wrist, namely, under the skin tissues of the front portion of the wrist, is damaged when the carpal tunnel gets narrower or internal pressure is increased due to various causes so that pain, numbness, and hypoesthesia may be caused in the palm and the fingers, which are innervated regions.

In order to protect the wrist where the carpal tunnel syndrome may occur, a wrist guard is used. Korean Patent Laid-open No. 10-2021-0023390 discloses a “wrist guard”. Referring to FIG. 1, the conventional wrist guard will be described.

FIG. 1 is a photograph showing a conventional wrist guard. Referring to FIG. 1, the conventional wrist guard includes: a protection part 10 having elasticity to wrap the outside of the user's wrist; a wearing part 20 connected to one side of the protection part 10 to wrap the outside of the protection part 10 and apply pressure; a circulation part 30 provided in the protection part 10 and the wearing part 20 or one portion thereof to allow the circulation of air while discharging moisture such as sweat generated from the wrist inside the protection part 10 to the outside; and an attachment means 40 having Velcro parts 41 and 42 to be detachably attached to the wearing part 20 while wrapping the protection part 10.

The conventional art having the above configuration discharges moisture to the outside through the circulation part 30 when a user is worn by exercise or movement.

However, the conventional wrist guard is weak in reinforcing force since the protection part 10 and the wearing part 20 surrounding the periphery of the wrist are made of an elastic material such as a fabric. Additionally, the conventional wrist guard is inconvenient in bending the wrist since the protection part 10 and the wearing part 20 surround the entire wrist including the temporal bone.

Patent Document 1: KR 10-2021-0023390 A (03.04.2021)

### SUMMARY

Accordingly, the present invention has been made to solve the above-mentioned problems occurring in the prior arts, and it is an object of the present invention to provide a wrist guard which intensively reinforces the wrist tunnel and the periphery of the ligament covering the wrist tunnel which causes pain when the wrist is bent, thereby enhancing a reinforcing force to the wrist of a user and preventing inconvenience when the user bends the wrist.

To accomplish the above object, according to the present invention, there is provided a wrist guard including: a compression part having elasticity and surrounding the periphery of the wrist; a band part disposed on the outer side of the compression part to surround and tighten the circumference of the compression part surrounding the wrist; and a reinforcing bar disposed between the band part and the compression part, located around the ligament covering the wrist tunnel, and extending toward the elbow.

Here, the band part includes: a wrist support surrounding around the ligament covering the wrist tunnel; and a strip extending from both ends of the wrist support to surround and tighten the circumference of the compression part surrounding the wrist, wherein the strip is narrower than the wrist support.

Moreover, the wrist support further comprises an extension portion convexly extending toward the elbow.

Furthermore, the strip has a ring disposed at the front end and a Velcro tape inserted into the ring and fastened to the ring.

Additionally, the base end of the strip surrounds and tightens between the wrist joint and the ulnar head.

In addition, preferably, the strip has a plurality of magnets disposed in the longitudinal direction of the strip.

Moreover, the reinforcing bar is disposed between the band part and the compression part in such a way that a pair of rectangular plates are located at both sides of the wrist tunnel.

Furthermore, preferably, the reinforcing bar has a plate shape that a plurality of protrusions and a plurality of valleys extend toward the elbow.

The wrist guard according to the present invention can increase a reinforcing force supporting the ligament since the wrist support, which intensively reinforces the wrist tunnel and the periphery of the ligament covering the wrist tunnel, has a wide area and the extension portion extending from the palm of a user toward the elbow is formed on the wrist support.

Furthermore, the wrist guard according to the present invention can stably tighten the wrist even though a user bends the wrist, and does not provide inconvenience in use since the strip of the band part fixing the compression part to tighten the user's wrist is located between the wrist joint and the ulnar head.

In addition, the wrist guard according to the present invention includes a pair of the reinforcing bars disposed between the compression part and the band part to be spaced apart from each other and the wrist tunnel is positioned between a pair of the reinforcing bars such that the reinforcing bars can firmly support the wrist without pressing the wrist tunnel and without inconvenience in bending the wrist when the user wears the wrist guard.

### BRIEF DESCRIPTION OF THE DRAWINGS

The above and other objects, features and advantages of the present invention will be apparent from the following detailed description of the preferred embodiments of the invention in conjunction with the accompanying drawings, in which:

FIG. 1 is a photograph showing a conventional wrist guard;

FIG. 2 is a perspective view illustrating a wrist guard according to a preferred embodiment of the present invention;

FIG. 3 is a plan view illustrating the wrist guard according to the preferred embodiment of the present invention;

FIG. 4 is an exploded perspective view illustrating the wrist guard according to the preferred embodiment of the present invention;

FIG. 5 is an enlarged cross-sectional view illustrating the wrist guard according to the preferred embodiment of the present invention;

FIGS. 6 and 7 are perspective views illustrating a state in which a user wears the wrist guard according to the preferred embodiment of the present invention;

FIG. 8 is a perspective view illustrating a reinforcing bar of a wrist guard according to another preferred embodiment of the present invention; and

FIG. 9 is a perspective view illustrating a strip of a wrist guard according to another preferred embodiment of the present invention.

### DETAILED DESCRIPTION

Terms and words used in the present specification and claims are not to be construed as a general or dictionary meaning, but are to be construed as meaning and concepts meeting the technical ideas of the present invention based on a principle that the present inventors may appropriately define the concepts of terms in order to describe their inventions in best mode.

Hereinafter, preferred embodiments of the present invention will be described in detail with reference to the accompanying drawings.

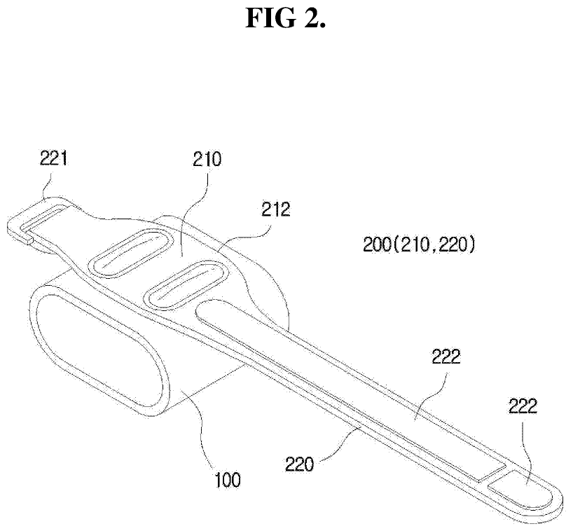

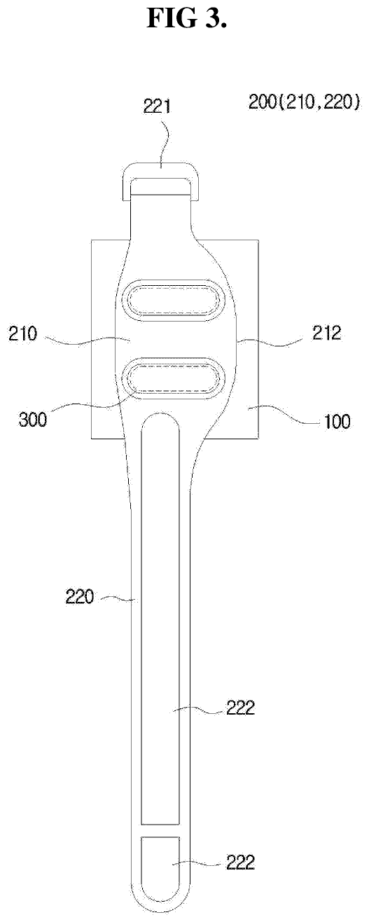

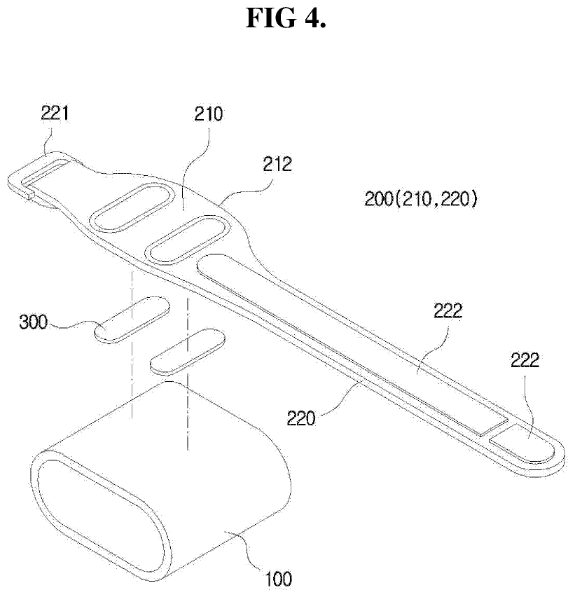

FIG. 2 is a perspective view illustrating a wrist guard according to a preferred embodiment of the present invention, FIG. 3 is a plan view illustrating the wrist guard according to the preferred embodiment of the present invention, and FIG. 4 is an exploded perspective view illustrating the wrist guard according to the preferred embodiment of the present invention.

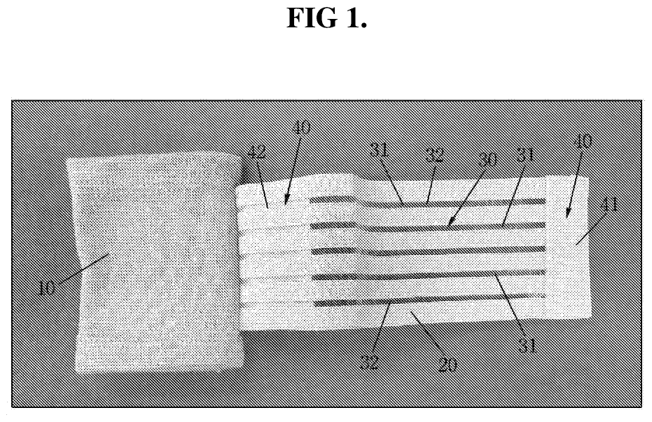

Referring to FIG. 1, the wrist guard according to the present invention includes: a compression part 100 worn on the wrist of a user; a band part 200 for tightening the wrist of the user from the outside of the compression part 100 to fix the compression part 100 to the wrist of the user; and a reinforcing bar 300 located around the ligament covering the wrist tunnel and positioned between the compression part 100 and the band part 200.

In other words, the compression part 100 has a cylindrical shape, is made of a fabric having elasticity so that the user can wear the wrist guard on the wrist. The compression part 100 which has elasticity comes into contact with the user's wrist to be suitable for the curve of the wrist, absorbs sweat generated from the wrist skin of the user, and is smoothly transformed according to the curve of the wrist, thereby minimizing a sense of difference in wearing.

The band part 200 is disposed outside the compression part 100 to surround the periphery of the compression part 100 surrounding the user's wrist. The band part 200 includes: a wrist support 210 which is attached to the outer surface of the compression part 100 to surround the periphery of the ligament covering the wrist tunnel; and a strip 220 extending from both ends of the wrist support 210 to surround the circumference of the compression part 100 surrounding the wrist.

The band part 200 may be integrally formed with the compression part 100 by being sewed at a portion getting in contact with the compression part 100, be integrally formed with the compression part 100 by an adhesive applied on the outer surface of the band part 200 getting in contact with the compression part 100, or be integrally formed with the compression part 100 by thermal bonding of the compression part 100 and the band part 200 since primer is interposed between the compression part 100 and the band part 200.

In addition, the strip 220 is provided with a ring 221 at the front end thereof and a Velcro tape 222 disposed at a base end thereof to be inserted into the ring 221. Accordingly, when the compression part 100 is put on the user's wrist, the front end and the base end of the strip 220 tighten the outer surface of the compression part 100 to fix the compression part 100 on the user's wrist.

The strip 220 is not limited to the ring 221 and the Velcro tape 222 described above, and may be transformed in various ways. For instance, the strip 220 may include a ring formed at the front end thereof, a retaining pin (not shown) disposed at the ring, and a plurality of holes (not shown) formed at the base end thereof in a longitudinal direction, so as to have a fixing structure that the retaining pin is inserted into the hole or a fixing structure that snap buttons are respectively disposed at the front end and the base end of the strip 220 to be coupled with each other.

The band part 200 has a structure thin waterproof films (not shown) are stacked on the upper surface and the lower surface of a cushiony member (not shown) showing an elastic restoring force like sponge. Accordingly, the band part 200 is not expanded in the longitudinal direction. When the front end and the base end of the strip 220 are coupled with each other to tighten the compression part 100, the cushiony member is elastically transformed according to the curve of the wrist, thereby minimizing a sense of difference and firmly fixing the compression part 100 on the wrist.

Especially, the band part 200 stiffens the reinforcing force on the user's wrist by intensively reinforcing around the wrist tunnel and the ligament covering the wrist tunnel causing pain when the wrist is bent after the compression part 100 of the wrist guard is worn on the user's wrist and is fixed on the wrist by the strip 220. The strip 200 is formed to be narrower than the wrist support 210 so as not to provide inconvenience in bending the wrist.

In other words, as illustrated in FIGS. 2 to 4, in case of the wrist support 210 and the strip 220, the strip 200 extending at both ends of the wrist support 210 is formed to be narrower than the wrist support 210.

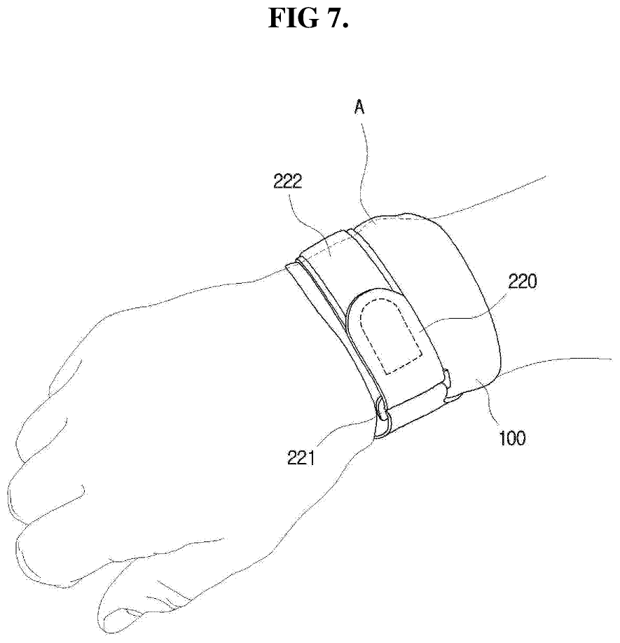

In this instance, as illustrated in FIG. 7, the base end of the strip 220 is as wide as to surround and tighten between the wrist joint and the ulnar head A so that the strip 220 can tighten the outer surface of the compression part 100. Accordingly, when the compression part 100 is fixed on the user's wrist, the base end of the strip 220 is located between the wrist joint and the ulnar head A so that the base end of the strip 220 is caught to the ulnar head A to stably tighten the wrist even though the wrist is bent. Therefore, the user does not feel inconvenience in a bending action of the wrist after wearing the wrist guard.

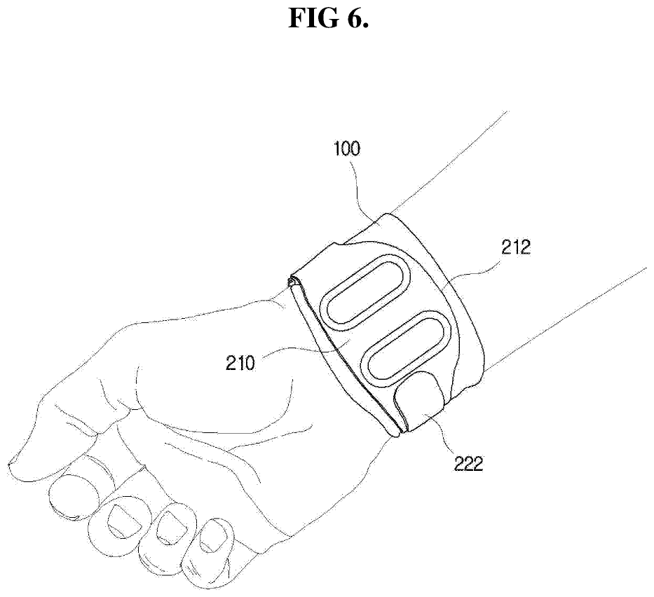

Moreover, the wrist support 210 has an extension portion 212 that extends convexly toward the elbow. As shown in FIG. 6, the extension portion 212 is extended from the inside of the wrist toward the elbow to increase the area of the wrist support 210. Furthermore, the extension portion 212 also increases the reinforcing force supporting the ligament covering the wrist tunnel due to an area increase of the wrist guard 210 by the extension portion 212 when the wrist is bent toward the palm.

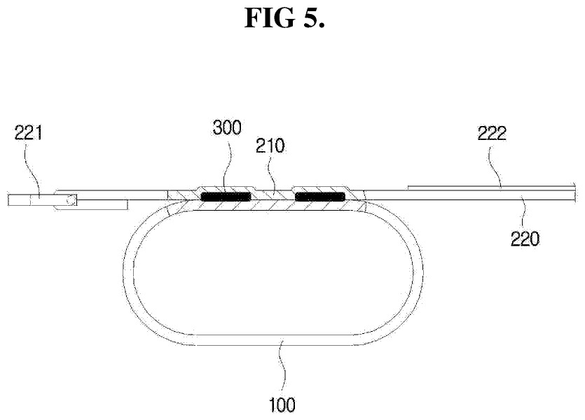

Meanwhile, the reinforcing bar 300 is disposed between the band part 200 and the compression part 100 to be located around the ligament covering the wrist tunnel. As shown in FIGS. 4 to 5, the reinforcing bar 300 is provided between the band part 200 and the compression part 100 such that a pair of rectangular plates are positioned at both sides of the wrist tunnel.

In this instance, the reinforcing bar 300 may be a pair of rectangular plates made of a hard material, such as synthetic resin or metal. Preferably, the reinforcing bar 300 is formed in a rectangular shape extending from the palm to the elbow, and the pair of reinforcing bars 300 are spaced apart from each other and the wrist tunnel is positioned between the pair of reinforcing bars 300.

As described above, when the pair of reinforcing bars 300 are spaced apart from each other and the wrist tunnel is positioned between the pair of reinforcing bars 300, the wrist guard according to the present invention does not provide inconvenience in bending the wrist and firmly supports the wrist since the reinforcing bars 300 do not press the wrist tunnel when the user wears the wrist guard.

The reinforcing bars 300 are not limited to the above-mentioned material, such as synthetic resin or metal, but may be modified in various ways. For instance, the reinforcing bar 300 may be made of a magnetic material generating a magnetic force, and when the reinforcing bar 300 is made of a magnetic material. In a case in which the reinforcing bar 300 is made of a magnetic material, it facilitates the blood flow of the wrist on which the reinforcing bar 300 is located, thereby helping health.

The wrist guard according to the present invention can increase a reinforcing force supporting the ligament since the wrist support 210, which intensively reinforces the wrist tunnel and the periphery of the ligament covering the wrist tunnel, has a wide area and the extension part 212 extending from the palm of a user toward the elbow is formed on the wrist support 210.

Furthermore, the wrist guard according to the present invention can stably tighten the wrist even though a user bends the wrist, and does not provide inconvenience in use since the strip 220 of the band part 200 fixing the compression part 100 to tighten the user's wrist is located between the wrist joint and the ulnar head A.

In addition, the wrist guard according to the present invention includes a pair of the reinforcing bars 300 disposed between the compression part 100 and the band part 200 to be spaced apart from each other and the wrist tunnel is positioned between a pair of the reinforcing bars 300 such that the reinforcing bars 300 can firmly support the wrist without pressing the wrist tunnel and without inconvenience in bending the wrist when the user wears the wrist guard.

The present invention is not limited to the embodiments. It will be understood by those skilled in the art that various changes and modifications may be made therein without departing from the technical idea and scope of the present invention, the changes and modifications belong to the spirit and scope of the present invention.

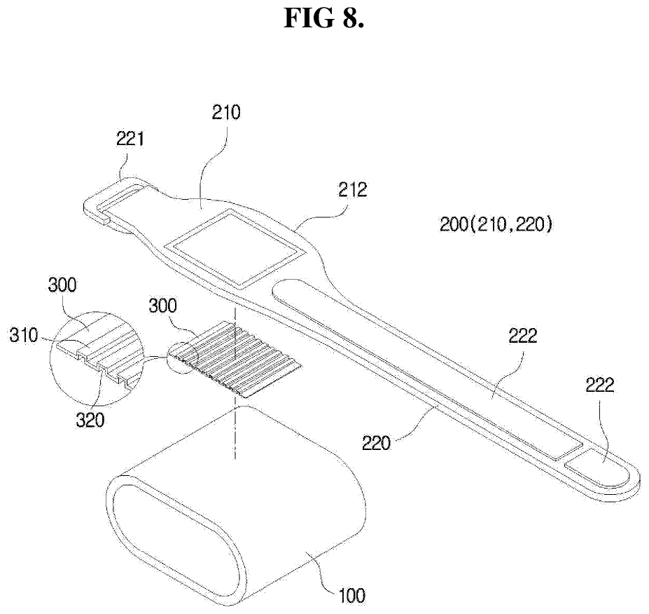

Although a pair of the reinforcing bars 300 provided between the compression part 100 and the band part 200 has been described as being spaced apart from each other around the wrist tunnel, as occasion demands, as shown in FIG. 8, the reinforcing bars 300 may be formed in the shape of a plate in which a plurality of protrusions 310 and a plurality of valleys 320 extend toward the elbow.

As described above, in a case in which the reinforcing bar 300 is formed in the shape of a plate in which a plurality of protrusions 310 and a plurality of valleys 320 extend toward the elbow, the reinforcing bar 300 is bent in the longitudinal direction of the strip 220 but is perpendicular to the longitudinal direction of the strip 220. That is, the reinforcing bar 300 is not bent in a direction toward the elbow from the palm of the user.

In a case in which the reinforcing bar 300 having the acid 310 and the bone 320 formed therein is provided between the compression part 100 and the band part 200, when the user wears the wrist guard, the reinforcing bar 300 surrounds the wrist while being bent around the wrist of the user, thereby firmly supporting the wrist without a sense of difference.

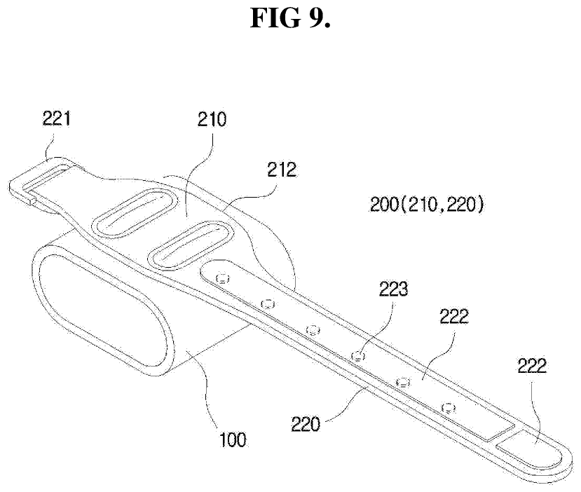

Moreover, as shown in FIG. 9, a plurality of magnets 223 may be provided inside the strip 220. That is, a plurality of magnets 223 are provided in the strip 220 in the longitudinal direction of the strip 220. The magnets 223 are in the form of a thin plate and are arranged in the longitudinal direction of the strip 220.

As described above, in a case in which the magnets 223 formed in the form of a thin plate is arranged in the strip 220, when the user wears the wrist guard, the magnets 223 disposed in the strip 220 are located along the circumference of the wrist, thereby facilitating the blood flow of the wrist by the magnetic force generated from the magnets 223 to help health.

What is claimed is:

1. A wrist guard comprising:
a compression part having elasticity and surrounding the periphery of the wrist;
a band part disposed on the outer side of the compression part to surround and tighten the circumference of the compression part surrounding the wrist; and
a reinforcing bar disposed between the band part and the compression part, located around the ligament covering the wrist tunnel, and extending toward the elbow.

2. The wrist guard according to claim 1, wherein the band part comprises:
a wrist support surrounding around the ligament covering the wrist tunnel; and
a strip extending from both ends of the wrist support to surround and tighten the circumference of the compression part surrounding the wrist, and
wherein the strip is narrower than the wrist support.

3. The wrist guard according to claim 2, wherein the wrist support further comprises an extension portion convexly extending toward the elbow.

4. The wrist guard according to claim 2, wherein the strip has a ring disposed at the front end and a Velcro tape inserted into the ring and fastened to the ring.

5. The wrist guard according to claim 2, wherein the base end of the strip surrounds and tightens between the wrist joint and the ulnar head.

6. The wrist guard according to claim 1, wherein the reinforcing bar is disposed between the band part and the compression part in such a way that a pair of rectangular plates are located at both sides of the wrist tunnel.
